(19) United States
(12) Patent Application Publication
(10) Pub. No.: US 2017/0311836 A1
(43) Pub. Date: Nov. 2, 2017

(54) System and Method for Wave Interference Analysis and Titration

(71) Applicant: Brigham and Women's Hospital, Inc., Boston, MA (US)

(72) Inventor: Michael O. Sweeney, Newton, MA (US)

(21) Appl. No.: 15/528,445

(22) PCT Filed: Nov. 20, 2015
(86) PCT No.: PCT/US15/61746

§ 371 Date: May 19, 2017

Related U.S. Application Data
(60) Provisional application No. 62/082,334, filed on Nov. 20, 2014.

(51) Int. Cl. A61B 5/0472; A61B 5/0408; A61N 1/368; A61B 5/00; A61N 1/365

(52) U.S. Cl. CPC A61B 5/0472; A61N 1/36507; A61B 5/4836; A61N 1/36843; A61B 5/0028; A61B 5/0408

(57) ABSTRACT

A system for cardiac monitoring and therapy includes a mother device configured to receive signals indicative of cardiac electrical activity in a patient's heart. The mother device includes a mother wireless communications module configured to transmit and receive information to d and from the mother device. The system also includes a satellite device configured to receive the signals indicative of the cardiac electrical activity in the patient's heart from a remote location relative to the mother device and includes a satellite wireless communications module configured to transmit from and receive communications sent to the satellite device to at least communicate with the mother wireless communications module. The system also includes a processor configured to receive the signals indicative of the cardiac electrical activity in the heart received by the mother device and the satellite device and, based thereon, control delivery of electrical therapy to the patient's heart.

### CROSS-REFERENCE TO RELATED APPLICATIONS

This application is based on, claims the benefit of, and incorporates herein by reference U.S. Provisional Patent Application Ser. No. 62/082,334, filed on Nov. 20, 2014, and entitled “System and Method for Automated Adjustment of Cardiac Resynchronization Therapy Control Parameters.”

### BACKGROUND

The present disclosure relates to systems and methods for cardiac monitoring and stimulation. More particularly, the present disclosure relates to systems and methods for timed electrical stimulation, or pacing, to generate improvement in cardiac structure and function.

Electrical therapies are targeted toward patients who have heart failure associated with cardiac timing abnormalities. This is due to optimal cardiac pump function depending on a condition of methodical arrangement of component parts that is precisely and dynamically orchestrated by electrical timing. This electromechanical ordering occurs at multiple anatomic levels, including within atria, between atria and ventricles, between ventricles, and especially within the left ventricle. Improper electrical timing disrupts these systematic arrangements, can occur in isolation or in various combinations at any anatomic level, and degrades cardiac pump function.

Therefore, it would be desirable to have additional systems and methods for providing electrical therapies and ensuring proper cardiac function.

### SUMMARY

The disclosure provides a system and method for monitoring cardiac activity and providing cardiac electrical therapy is provided. The system includes a mother device and a separate, physically remote, implanted satellite device. Together the mother device and satellite device create a wireless network to share information and determine QRS fusion wave information that can be used to control electrical therapy.

In accordance with one aspect of the disclosure, a cardiac implantable electrical system is provided for monitoring and delivering electrical therapy to a patient's heart. The system includes a mother device having electrodes configured to positioned to extend through the patient to receive signals indicative of cardiac electrical activity in the heart, an impulse delivery system for delivering electrical impulses to the heart to provide cardiac electrical therapy thereto, and a mother wireless communications module configured to transmit and receive information to and from the mother device. The system also includes a satellite device separate from the mother device and configured to be implanted remotely from the mother device to receive the signals indicative of the cardiac electrical activity in the heart from a remote location relative to the mother device and a satellite wireless communications module configured to transmit from and receive communications sent to the satellite device to at least communicate with the mother wireless communications module. The system also includes a processor configured to receive the signals indicative of the cardiac electrical activity in the heart received by the mother device and the satellite device. The processor is programmed to compare the signals indicative of the cardiac electrical activity in the heart to the baseline electrical activity to determine a QRS fusion wave contour, characterize an electrical activation sequence of the heart of the patient using the QRS fusion wave contour, and control the impulse delivery system based on the characterized electrical activation sequence of the heart.

In accordance with another aspect of the disclosure, a system is provided for delivering cardiac therapy to a patient's heart with a cardiac rhythm management (CRM) device. The system includes a satellite device configured to receive signals indicative of the cardiac electrical activity in the heart from a remote location relative to the heart and including a satellite wireless communications module configured to transmit from and receive communications sent to the satellite device. The system also includes a mother device having electrodes configured to positioned to extend through the patient to receive signals indicative of cardiac electrical activity in the heart, a memory storing electrical therapy parameters, an impulse delivery system for delivering electrical impulses to the heart to provide cardiac electrical therapy thereto based on the electrical therapy parameters, and a mother wireless communications module configured to transmit and receive information to and from the mother device with at least the satellite wireless communications module. The mother device also includes a processor configured to receive the signals indicative of the cardiac electrical activity in the heart received by the mother device and the satellite device. The processor is programmed to compare the signals indicative of the cardiac electrical activity in the heart to the baseline electrocardiograph electrical activity to determine a QRS fusion wave contour, characterize an electrical activation sequence of the heart of the patient using the QRS fusion wave contour, and adjust the electrical therapy parameters based on the characterized electrical activation sequence of the heart.

In accordance with yet another aspect of the disclosure, a cardiac implantable electrical system for monitoring and delivering electrical therapy to a patient's heart. The cardiac implantable electrical system includes a mother device configured to receive signals indicative of cardiac electrical activity in a patient's heart and including a mother wireless communications module configured to transmit and receive information to and from the mother device. The system also includes a satellite device configured to receive the signals indicative of the cardiac electrical activity in the patient's heart from a remote location relative to the mother device and including a satellite wireless communications module configured to transmit from and receive communications sent to the satellite device to at least communicate with the mother wireless communications module. The system further includes a processor configured to receive the signals indicative of the cardiac electrical activity in the heart received by the mother device and the satellite device and, based thereon, control delivery of electrical therapy to the patient's heart.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a graphic depiction of electrical wave interference superposition and nomenclature in accordance with the present disclosure.

FIG. 2 is a graphic depiction of waves representing typical QRS complex interactions between an exemplary right ventricular (RV) monochamber wavefront (QS waveform complex), an exemplary left ventricular (LV) monochamber wavefront (R, Rs waveform complex), and a plurality of resulting multipoint electrical stimulation wave interference products from the viewpoint of leads V1-V2, in accordance with the present disclosure.

FIG. 3 is a graphic depiction of the QRS fusion wave contour showing that, as used in accordance with the present disclosure, the QRS fusion wave contour is visually distinctive, mathematically quantifiable, and demonstrably able to characterize base condition electrical activation times, change in electrical activation times in response to single or multipoint wavefront fusion, and change in wave force directionality and magnitude, which are collectively used to characterize the cardiac electrical activation sequence.

FIG. 4 is a block diagram illustrating a system and method for applied QRS fusion wave analysis in accordance with the present disclosure.

FIG. 5 is a pictorial representation of a system for a wearable subcutaneous local area network for wave interference analysis and titration.

FIG. 6 is a pictorial representation of a mother component of the system of FIG. 5.

FIG. 7 is a pictorial representation of a satellite component of the system of FIG. 5.

FIG. 8 is another pictorial representation of a satellite component of the system of FIG. 5.

FIG. 9 is a pictorial representation of another system for a wearable subcutaneous local area network for wave interference analysis and titration.

### DETAILED DESCRIPTION OF THE INVENTION

Conventional cardiac pacing with implanted cardiac rhythm management (“CRM”) devices, such as pacemakers and implantable cardioverter-defibrillators (“ICDs”) with pacing functionality, involves delivering electrical pacing pulses to a patient's heart via intracardiac electrodes that are in electrical contact with desired portions of the heart. The CRM device is usually implanted subcutaneously on the patient's chest.

Conventional cardiac implantable electrical devices (“CIED”) includes two basic components. First, the CIED includes an implantable pulse generator. Second, the CIED includes an intracardiac lead system that may extend into the patient's heart by way of the vessels of the upper venous system, such as the superior vena cava; or other methods of access to the heart. The pulse generator is designed to detect cardiac arrhythmias and/or pacing or defibrillation conditions and deliver therapy in the form of electrical stimulation pulses or shocks delivered to the heart through the lead system.

The present disclosure breaks from this traditional paradigm of CIED by providing for the controlled initiation and propagation of single or multiple opposing radial wavefronts generated by artificial pacemaker stimulation and fused with spontaneous cardiac electrical activity to normalize cardiac electromechanical activation. The sum of these electrical waves is expressed on the body surface as the QRS complex, which is characterized and quantified using the method of wave interference superposition between two or more point sources (“fusion”). That is, the present disclosure recognizes that a QRS fusion complex can be utilized for grading electrical wave fusion efficiency, evaluating prospective electrical stimulation sites and wave interactions, plotting wave propagation paths, and informing wave timing instructions. Furthermore, the present disclosure recognizes that the QRS fusion complex is mathematically quantifiable and demonstrably suitable for multivariable clinical event prediction models. Thus, a system and method is provided for leveraging these concepts to provide an improved CIED system and architecture that is capable of providing therapies not contemplated by traditional CIED systems.

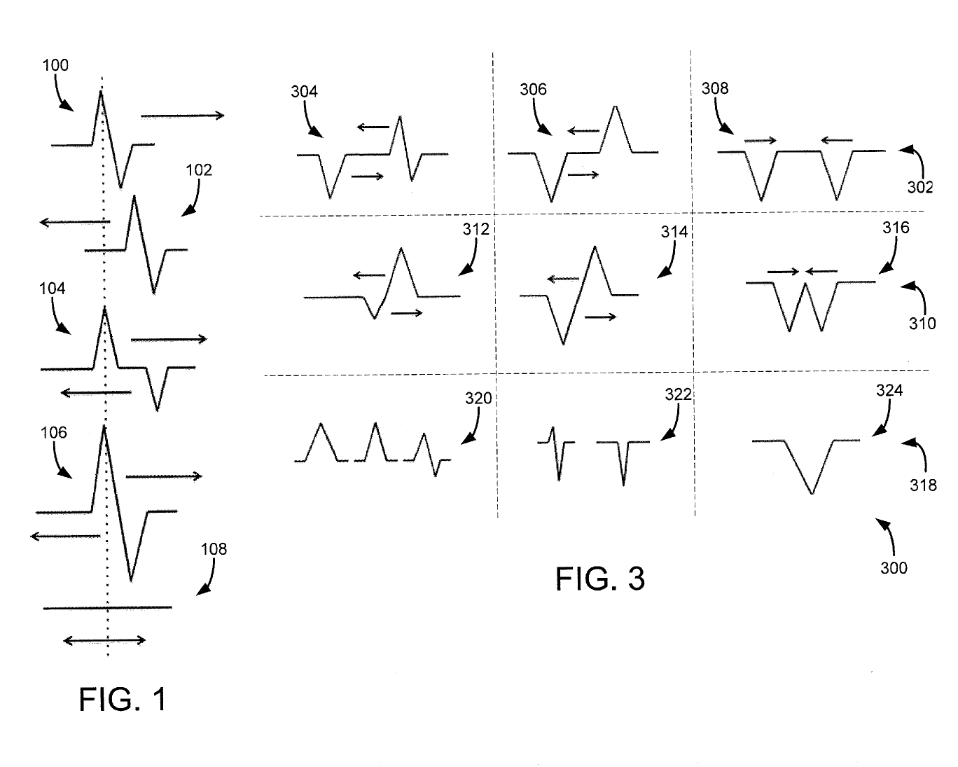

Referring to FIG. 1, a depiction of electrical wave interference superposition and nomenclature as related to the present disclosure is provided. First a “rightward wave” 100 is illustrated. Next, a “leftward wave” 102 is illustrated. When two waves come together at the same point, they interfere with each other. If the waves 102, 104 are in phase with each other, peaks align and troughs align, wave forces add; this is constructive interference. If the waves are out of phase, peaks align with troughs, wave forces subtract or cancel; this is destructive interference. Interference determines the quantifiable physical characteristics of the resulting wave, such as shape, amplitude, and duration.

At the first point of interaction, the negative component (trough or downward displacement) of the rightward wave 100 and the positive component (peak or upward displacement) of the leftward wave 102 are precisely aligned and cancel, yielding a combined fusion wave contour forming a partial destructive interference wave form 104 that is intermediate in shape between the two independent interfering waves 100, 102, termed conformational change.

At the second point of interaction, the positive (peaks) and negative (troughs) displacements of both waves reinforce to create a constructive interference waveform 106. In the constructive interference waveform 106, the wave contour duplicates independent interfering waves, but with increased amplitudes.

At the third point of interaction, the reinforced positive (peak) and negative (trough) displacements are perfectly aligned and completely cancel (zero amplitude), represented by the complete destructive interference waveform 108.

In accordance with the present disclosure, normal spontaneous cardiac electrical activation occurs as a consequence of widespread wave cancellation among a multitude of interfering wavefronts distributed by the specialized conduction system. As will be described, this phenomenon can be closely simulated by two or more opposing radial wavefronts generated by electrical stimulation and fused with spontaneous electrical waves to correct disordered electrical wave propagation and restore normal cardiac electrical activation. The result is that the reconstituted muscle contraction wave synchronizes all points of cardiac electromechanical activation.

Thus, a fusion wavefront is a composition of the individual wavefronts and is influenced by the phase relationship, shape, amplitudes, and durations of the individual wavefronts. Phase relationship is influenced by stimulation timing (such as simultaneous vs. sequential stimulation), wave propagation path which in turn is directly influenced by stimulation site, and barriers to wavefront propagation (such as scar volume or other forms of conduction block) that alter the propagation path. Generally, a consequence of high wavefront coherence is that the relative phase relationship between wavefronts is constant, or fixed. However, it is possible that some dynamic variation in wavefront behavior could occur due to fluctuation in conduction properties of the intervening myocardial tissue. Furthermore, as will be described, the wavefront phase relationship can be altered by manipulation of pacing control parameters that influence chamber timing, in particular, ventricular activation timing. Thus, a fusion wavefront can serve as a biomarker and can be manipulated by pacing control parameters to achieve specific, desired patterns correlated with increased odds of clinical improvement (specifically, reverse remodeling) and tailored to the individual patient.

A de novo conceptual framework using wave mechanics is provided to analyze wavefront propagation for general recognition of effective or ineffective ventricular activation wavefront fusion response is provided in co-pending U.S. application Ser. No. 14/131,868, entitled System and Method for Automated Adjustment of Cardiac Resynchronization Therapy Control Parameters, which is incorporated herein by reference in its entirety. This framework provides a construct for characterizing specific activation patterns during left bundle branch (LBBB) and cardiac resynchronization therapy (CRT). In this construct, CRT illustrates an example of high coherence wavefront interference between individual advancing wavefronts from two or more sources or stimulation sites (such as the right ventricle and the most electrically delayed segment of the left ventricle during biventricular pacing). Taking this a step further than a simple effective/ineffective observation, exhibited wavefront interference characteristics can be differentiated into three distinct, mutually exclusive and encompassing ventricular activation response pattern phenotypes to CRT (an oblique fusion responder phenotype, a cancellation fusion responder phenotype, and a summation fusion non-responder phenotype), as further described below.

As described above, a fusion wavefront is a composition of individual wavefronts and is influenced by the phase relationship and amplitudes of the individual wavefronts. If the propagating wavefronts are opposed, the resulting activation pattern of the fusion wavefront reflects a destructive interference. In this usage, destructive interference means that two advancing opposing wavefronts tend to cancel one another out, to varying degrees, by subtraction. Thus, in this case, a baseline ventricular activation sequence signature of LBBB will be replaced by an effective paced ventricular activation sequence of admixtures of the variably opposed advancing wavefronts, which cancel one another. In this usage, effective means an activation sequence that reduces ventricular conduction delay relative to LBBB by the method of wave cancellation, a necessary requirement for improvement in left ventricle (“LV”) pump function.

In contrast, the observation of an exaggeration of the LBBB baseline activation sequence during CRT is characterized by constructive interference. In this usage, constructive interference means that the two advancing wavefronts enhance one another by summation, and therefore worsen the baseline ventricular conduction defect. The wavefronts are non-oppositional. This form of ineffective paced ventricular activation sequence, or fusion failure, does not reduce ventricular conduction delay relative to LBBB, thereby eliminating the possibility of an improvement in LV pump function. Also, in some cases, constructive interference may enhance, or reinforce, the underlying ventricular conduction delay (wave summation).

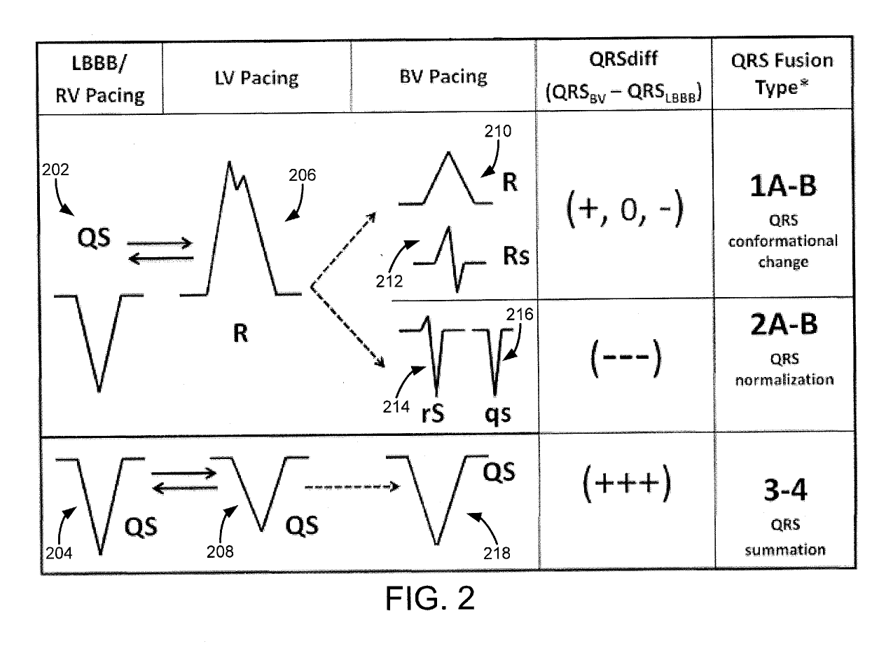

Referring now to FIG. 2, a graphic table is provided that shows the relationship between different pacing to fusion types, in accordance with the present disclosure. For this non-limiting example, assume a LBBB activation displays typical QS waveform complex. Nine general patterns, or QRS categories are identified, as described below in Table 1.

TABLE 1

| Category | Description |
|---|---|
| R | Only R-wave present |
| RS | R-wave and S-wave present with equal amplitude |
| Rs | R-wave and S-wave present, R-wave with greater amplitude |
| rS | R-wave and S-wave present, S-wave with greater amplitude |
| QS | Q-wave and S-wave present with equal amplitude |
| qR | Q-wave and R-wave present, R-wave with greater amplitude |
| QR | Q-wave and R-wave present with equal amplitude |
| Qr | Q-wave and R-wave present, Q-wave with greater amplitude |
| QRS | Q-wave, R-wave, and S-wave are all present |

As shown in FIG. 2, these general categories described in Table 1, can be combined to create QRS fusion types. Specifically, as a non-limiting example, LBBB/RV pacing having a given QS wave 202, 204 can be correlated with LV pacing of, for example, an R or QS wave 206, 208, respectively. The R wave 206 can be decomposed into an R wave 210 and an Rs wave 212 or an rS wave 214 and a qs wave 216 for the baseline ventricular (BV) pacing. On the other hand, the QS wave 208 corresponds to a QS wave 218 for the BV pacing. Based on this understanding, a QRS difference (QRS_{diff}), which in this example is difference between the QRS BV (QRS_{BV}) and the QRS LBBB (QRS_{LBBB}), can be calculated.

That is, patterns can be characterized by analyzing relative phase relationship (relative timing of wavefront peaks and troughs) and relative wave amplitudes of propagating wavefronts in comparison to a baseline wavefront sequence. In addition, a difference calculation between the QRS durations (“QRSd”) of the paced waveform and the baseline waveform can reflect the phase relationship and relative amplitudes of the waveforms, as well as quantify the change in total ventricular electrical asynchrony. This difference calculation, termed “QRSdiff” (or alternatively, “QRSdec”), can be derived from a 12 lead ECG according to the formula:

QRSdiff=(pQRSd)−(bQRSd)

Baseline QRSd (“bQRSd”) is a measure of total baseline ventricular electrical activation time (“bVAT”). Paced QRSd (“pQRSd”) is a measure of total paced ventricular activation time (“pVAT”). Therefore, QRSdiff is a measure of the difference in total ventricular activation (“VAT”) before and after CRT. When the pQRSd is shorter than the bQRSd, the QRSdiff is negative (less than zero), indicating a reduction in total ventricular electrical asynchrony and more efficient electrical resynchronization. An increasingly negative QRSdiff indicates a greater correction of the baseline ventricular conduction delay and is associated with increased odds of reverse remodeling, relative to a less negative QRSdiff value. In contrast, when the pQRSd is longer than the bQRSd, the QRSdiff is positive (greater than zero), indicating an increase in total ventricular electrical asynchrony and less efficient electrical resynchronization, which may be correlated with reduced odds of reverse remodeling (in combination with additional evidence, such as absence of a positive change in ventricular activation sequence, as further described below). In addition, accordingly, a neutral, or zero value QRSdiff indicates no change in VAT. QRSdiff may also be used as a change measure for baseline LV conduction delay in response to CRT.

In the present example, a BV pacing represented by the R wave 210 and Rs wave 212 corresponds to a QRS difference of (+, 0, −) and a QRS fusion type of 1A-B, which represents QRS conformational change. On the other hand, for the rS wave 214 and qs wave 216, the QRS difference is (−, −, −) and the QRS fusion type is 2A-B, which represents QRS normalization. Finally, the QS wave 218 corresponds to a QRS difference of (+, +, +) and a QRS fusion type of 3-4, which represents QRS summation.

As illustrated, QRS fusion wave contours and change in electrical activation times (QRSdiff) are complementary markers for change in cardiac activation wave forces and electrical resynchronization. Greater wave force normalization indicates a larger portion of electrical wave forces have been resynchronized, generating a large cancellation effect for the same reduction in electrical activation time. The QRS fusion waveform is a visual and quantifiable indicator of the volume of working myocardium that is electrically resynchronized. Therefore, in accordance with the present disclosure these characteristics can serve as a biomarker that provides electrocardiographic imaging of the electromechanical coupling of the heart.

The QRS fusion wave contour, as used in accordance with the present disclosure, is visually distinctive, and mathematically quantifiable. As will be described, the QRS fusion wave contour is demonstrably able to characterize base condition electrical activation times, change in electrical activation times in response to single or multipoint wavefront fusion, and change in wave force directionality and magnitude, which, in accordance with the present disclosure, can be used to characterize the cardiac electrical activation sequence.

Referring to FIG. 3, some additional non-limiting example waves 300 are depicted to represent traditional QRS complex interactions between an exemplary right ventricular (RV) monochamber wavefront (QS waveform complex), an exemplary left ventricular (LV) monochamber wavefront [symbol] R, Rs waveform complex), and a plurality of resulting multipoint electrical stimulation wave interference products from the viewpoint of leads V1-V2, as related to the present disclosure.

For exemplary purposes, again assume a LBBB activation displays a typical QS or rS waveform complex in the idealized recording location on the chest wall. Note, wave shapes, amplitudes, and durations vary and influence the multipoint fusion wavefront interference product. More particularly, in the upper-most row of waveforms 302 in FIG. 3, RV pacing generates QS waveform complex (moving right→left and anterior→posterior across the cardiac volume), lateral LV pacing generates R waveform complex (moving left→right and posterior→anterior across the cardiac volume). Thus, the non-limiting example waves 300 show modestly oppositional waves 304, highly-oppositional waves 306, and non-oppositional waves 308. More particularly, modestly-oppositional waves 304 show that the wave shapes are dissimilar, amplitudes differ, and wave forces are somewhat opposing. For the highly-oppositional waves 306, wave shapes and amplitudes are nearly identical and forces are directly opposing. Finally, for the non-oppositional waves 308, wave shapes and amplitudes are identical and wave forces are reinforcing because the LV wavefront is generated by a stimulation site or technique that does not oppose either RV pacing wave forces or LBBB wave propagation path.

In the middle row of waveforms 310 in FIG. 3, onset of fusion results in conformational change in the QRS complex, showing recognizable elements of the independent interacting waves. In particular, a mixed interference QRS fusion type 1 wave 312 shows the qR waveform complex, a mixed interference QRS fusion type 1 wave 314 shows the QR waveform complex 2, and a constructive ARS fusion type 3-4 wave 316 shows the QrS waveform complex.

Finally, in the bottom row of waveforms 318, a constructive/destructive interference − or + cancellation shows the final fusion QRS complex is R, Rs, rS, etc. (conformational hybrid). Due to this mixture of constructive and destructive interference, amplitudes and duration are reduced vs. baseline waveforms, if destructive interference (cancellation) dominates. These are exemplary forms of QRS Type 1 (QRS type 1A and 1B) (new R wave, with or without reduction in QRS activation time) as compared to LBBB. Also, a destructive interference +++ cancellation wave 322 shows the final fusion complex is qs (QRS normalization, a non-conformational change indicated by a large degree of wave force normalization). The destruction of both R wave and QS waves (wave suppression) and marked reduction in wave duration is present vs. baseline waveforms due to large, dominant cancellation effect. This is an example of QRS Type 2 (QRS type 2A and 2B). Finally, a constructive interference—non-cancelling wave interaction 324 shows the final fusion complex is QS. The QS complex is increased in amplitude and duration due to dominant constructive interference effect, indicating wave force summation. This is an example of QRS Types 3-4. Wave force summation may occur when LV stimulation originates from a non-opposing site to LBBB, or when high grade conduction block prevents propagation of an oppositional wave, or when wave timing parameters are incorrectly adjusted.

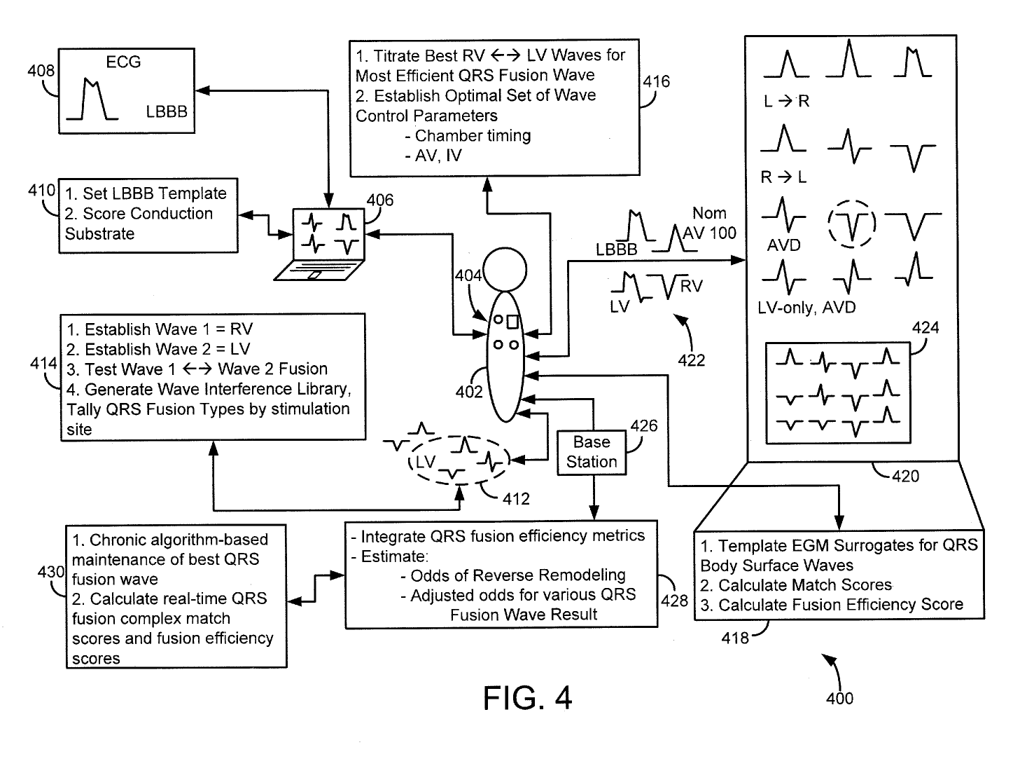

Referring now to FIG. 4, a block diagram is provided illustrating a system 400 and method for using the system to perform QRS fusion wave analysis in accordance with the present disclosure, the system 400 is designed to determine a QRS wave contour under different states and use it to characterize base condition electrical activation times, base condition myocardial scar, change in electrical activation times in response to single or multipoint wavefront fusion, and change in wave force directionality and magnitude, which, in accordance with the present disclosure, can be used to characterize the cardiac electrical activation sequence. The system 400 is designed to acquire information from a subject 402 that, in accordance with the present disclosure, is coupled to an ECG monitoring system and may have a wearable subcutaneous local area network 404. Regardless of the particular feedback system, cardiac waveforms may be gathered by a workstation 406. The workstation 406, may facilitate a patient selection process 408. The patient selection process 408 can use the conventional 12-lead ECG to identify LBBB, or its surrogate in the form of obligatory RV electrical stimulation, as detailed below. In particular, LBBB is identified by specific features of time duration and morphology. The salient QRS morphologic features of LBBB include a monophasic QS complex, or rS complex in lead V1; monophasic R complex in lead V6 which may be accompanied by a Q wave, a delayed intrinsicoid deflection, and a monophasic R wave in lead I, which may be accompanied by a Q wave. The T wave is usually directed opposite the latter portion of the QRS complex. The QRS duration exceeds 120 milliseconds. A similar pattern is observed during obligatory RV electrical stimulation. Both LBBB and RV stimulation therefore have in common a prolonged QRS duration, right-to-left electrical wave propagation, greatest electrical delay over the left-sided ECG leads (I, L, V5-V6), prominent notching indicating conduction block and delay, and a ST segment sloping off to T wave in the direction opposite the main QRS deflection.

Once the patient selection process is complete 408, the workstation 406 can be used to perform substrate-wave quantification 410. The process begins by selecting a template (per the above non-limiting example, the template may be an LBBB template), which allows scoring of the patient-specific electrical conduction substrate. Such scoring includes quantification of LV electrical delay and myocardial substrate in LBBB. LV electrical delay, quantified by QRS notching, displays a linear mathematical relationship with QRS duration. Whereas, the QRS duration in LBBB is used to identify patients for CRT, it is the LVAT_{MAX} that is the target of ventricular conduction replacement. Minimization of LVAT_{MAX} by QRS wave fusion is the mechanism of reverse remodeling. Prior work revealed that LVAT_{MAX} can be obtained from analysis of the QRS complex during LBBB. The numeric relationship between QRSd and LVAT_{MAX} was derived with the use of linear regression, where (LVAT_{MAX} [ms]=−35.839+0.763×QRSd [ms]+0.000619×QRSd [ms] [symbol] 2).

With this setup complete, the wavefronts can be analyzed 412 to facilitate an implant guidance process 414. The implant guidance process 414 begins with establishing a first wave in the RV and then establishing a second wave in the LV. The spontaneous RV wave duplicates the baseline LBBB wave in the absence of atrioventricular block. In the absence of spontaneous ventricular conduction (e.g., presence of atrioventricular block), the RV wave is necessarily generated by RV electrical stimulation. Both RV waveform origins are suitable for QRS fusion titration and analysis. In particular, the spontaneous RV wave, when present, and in the absence of prohibitive AV block, is suitable for achieving QRS fusion when combined with LV-only stimulation. That is, QRS fusion between spontaneous ventricular conduction during LBBB and LV-stimulation unaccompanied by RV stimulation. More commonly, QRS fusion is generated by conventional biventricular (BV) stimulation where both the RV wave and LV wave are initiated by separate but cooperatively timed electrical stimulation. The LV wave is highly sensitive to stimulation conditions, specifically the physical location of the stimulation electrode on the epicardium or endocardium of the left ventricle. Typically, LV stimulation is achieved by an electrode positioned within a coronary vein on the epicardial surface of the left ventricle. Such electrodes commonly have 2 (“bipolar”) or more (multipolar, or “quadripolar”) electrodes that allow for multiple stimulation paths that directly influence the physical and electrical characteristics of the LV wave, and thereby directly influence the QRS fusion wave. Such flexibility in LV wave generation can be exploited to identify a LV wave that is highly oppositional to the RV wave, which is generally fixed. Oppositional measures may include morphology, amplitude, time duration, and point-by-point numerical analysis of the wavefront contour. In this manner, a detailed survey of stimulation sites and electrical conduction pathways can be exploited to build a patient-specific wave interference library of QRS fusion products across a range of stimulation sites and wave propagation pathways.

With this setup process complete, acute QRS fusion titration can be performed 416. For example, wave control parameters that influence chamber timing, such as the atrioventricular timing interval, the interventricular timing interval, in combination with single or multisite LV stimulation and propagation path manipulation, can be used to identify the optimal set optimal set of wave control parameters. Such titration can be applied relative to the best RV/LV waves to yield the most efficient QRS fusion wave interference product.

Thereafter, chronic QRS fusion titration, optimization and maintenance (e.g., closed loop) can be performed 418. That is, upon identifying the most efficient QRS fusion complex for a given set of timing and stimulation site conditions, this complex is used as a template for matching ongoing QRS fusion complexes during continuous delivery of therapy. This optimized QRS fusion complex template can be directly recorded from one or more subcutaneous signal sources, such as a local subcutaneous network, and/or recorded from intracardiac electrograms, or from body surface recording sources, wherein the controller is directly, or indirectly (via telemetry link with a receiving device) receiving conventional electrocardiographic signals. These complementary signal sources for body surface waves 420 can be used to calculate QRS fusion complex match scores and fusion efficiency scores. The result of template selection is a set of templates 422 that can be used and compared to 12-lead ECG data 424 to determine the best QRS fusion product.

A base station 426 may be used to perform a variety of processes and, more specifically, apply heart failure event prediction models and perform data integration 428. To do so, the base station 426 may determine substrate configuration, QRS fusion result (titrated), ΔVAT, LV stimulation state including differential propagation paths, and estimate odds of reverse remodeling, estimate odds of death, or adjust odds for various QRS fusion wave results. For example, patient-specific odds of reverse remodeling are accurately predicted by a unique multivariable regression model that recites the interaction between (1) QRS Fusion Type, (2) QRS difference (QRS_{diff}, in milliseconds=QRS_{BV}−QRS_{LBBB}, which is a ΔLVAT measure, see above) induced by the QRS fusion response and indicative of the efficiency in the reduction of electrical asynchrony induced by wave cancellation, (3) QRS score for LV scar (where more scar reduces remodeling odds). Here we understand that the QRS fusion type 1 is the statistically dominant variable anticipating reverse remodeling, where QRS Fusion Type 2 is most efficient, followed by Type 1 (intermediately efficient) followed by Type 3 (inefficient).

Based thereon, additional ambulatory and remote reporting processes may be performed 430, including methods for maintaining optimal QRS fusion on a cycle-to-cycle basis by comparing the incoming QRS fusion complexes with the optimal QRS fusion complex identified during titration. Incoming QRS fusion complexes may be admitted when the controller, or “mother” device, is coupled to an ECG monitoring system and, or, when the controller, or “mother” device is coupled to a wearable subcutaneous local area network. The mother device receives body surface QRS complex data from the subcutaneous local area network and/or conventional body surface ECG electrodes in communication with external systems, such as the above-described base station 406. This data stream is used to thereby used to characterize the QRS fusion response during real-time operation. Once the optimal QRS fusion wave is generated, the overarching goal is to maintain that electrical activation pattern on a beat-to-beat basis, analogous to the beat-to-beat constancy of the normal QRS in the healthy heart. That is, dynamic, algorithm-driven changes in chamber timing parameters must be constrained to prevent unintentional disruption of optimal QRS fusion. Therefore, boundary conditions may be imposed on chamber timing manipulations in order maintain optimal QRS fusion. Otherwise, remodeling odds may decline. In this manner, an individualized patient-specific QRS fusion titration library is composed and maintained to facilitate uninterrupted optimal QRS fusion.

Table 1 provides a representative schema for QRS fusion wave analysis in accordance with the present disclosure.

TABLE 1

| | Measure | Quantification |
|---|---|---|
| Before Implant | | |
| LBBB QRS analysis | LV muscle scar (% LV volume score) | QRS Score for LBBB |
| | LV electrical delay | LVATmax (milliseconds) |
| During Implant | | |
| Wave interference | Monochamber wavefront analysis | Wave boundary solution |
| | Survey stimulation sites (multipolar) | Maximize wave opposition |
| | QRS Fusion titration (wave timing) | QRS Type (1-4) |
| | Change in EAS (electrical asynchrony) | Δ EAS (VAT measure) |
| | QRS fusion wave efficiency analysis | QRS Type + ΔEAS + LV lead |
| | QRS fusion wave interference score | Tally cancellation markers |
| After Implant | | |
| Prediction model | Cardiac volume, structure; death | Odds Ratio, plots |

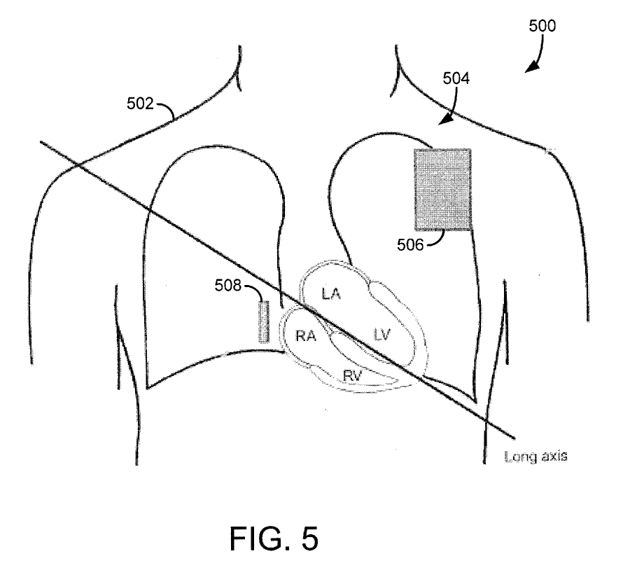

Referring now to FIG. 5, an example of a physical structure of a body-based communication network for QRS fusion wave analysis 500 is provided. In particular, a subject 502 is shown that has a point-to-point network topology 504 spatially distributed therethrough. The point-to-point network topology 504 is spatially distributed and autonomous, but with cooperating sensors, which jointly pass data through body tissue separating an implanted intra-body mother 506 device and a physically distant intra-body satellite 508 device. The use of body tissue for network communication establishes a body area network, and “body bus” for signal transmission.

The autonomous satellite device 508 designed to accurately duplicate the idealized body surface electrocardiographic viewpoints for characterizing and quantifying the cardiac electrical wave interference pattern, or QRS complex fusion response, as outlined above. The satellite device 508 is inserted simultaneously, or following, insertion of the compatible mother device 506. The housing is capable of recording a multiplicity of body surface electrical waveforms generated by cardiac electrical activity. The satellite device 508 may be seated on the chest wall in a subcutaneous (or submuscular) location capable of accurately characterizing left versus right sided cardiac electrical activity. This configuration enables the tracking of QRS fusion, as described above.

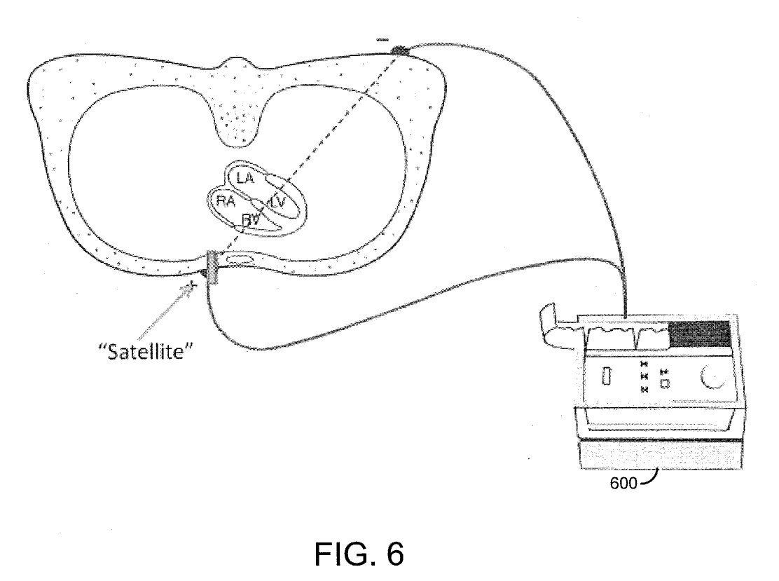

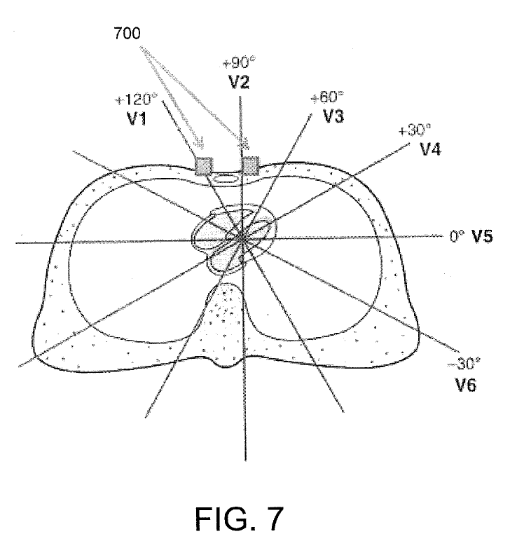

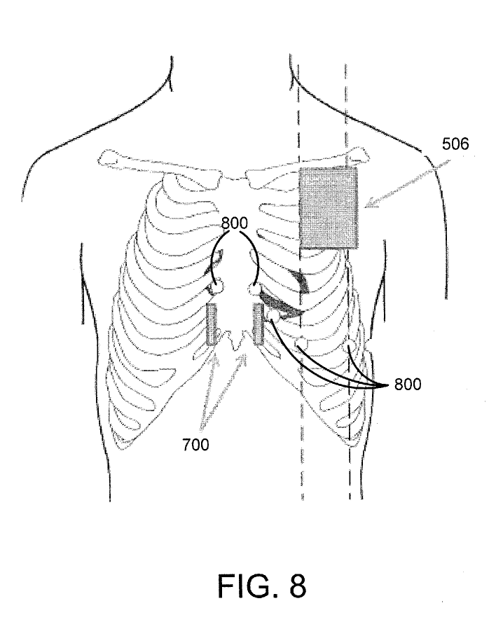

As described above, these wavefronts may be independent or codependent with spontaneous cardiac electrical waves, the goal being electrical wave resynchronization between right-sided and left-sided wave generating sources. The preferred recording sites 700 may vary between patients, but is typically located where the extensions of the short axis of the heart intersect with the body surface as illustrated in the horizontal plane view of FIG. 5 and FIG. 8 or the transverse view of FIG. 6 and FIG. 7. As illustrated in FIG. 7, this configuration duplicates the point of view of leads V1 and V2 in conventional 12 lead body surface electrocardiography, which prior work has shown to be ideal for assessing QRS wave fusion because these recording sites are aligned perpendicular to the interatrial and interventricular septa. This location 700 for the satellite device 508 is generally identified by the 4th intercostal space at the right or left edge of sternum, as illustrated in FIG. 8. In this arrangement the satellite device 508 is anatomically anterior to the heart and can, therefore, also be used to record and characterize additional wave signals between a second or a multiplicity of electrodes positioned at remote locations 800, such as the shell of the mother device 506, or more posterior electrodes positioned within or outside the cardiac surface. Such supplemental wavefront signals can be used to enhance body surface QRS fusion analysis and instruct electrical wave timing parameters.

The mother device 506 location may be unrestricted since primary signal acquisition for electrical wave analysis is achieved by the satellites device 508. Accordingly, the mother device 506 can be sited subcutaneously or submuscularly anywhere on the chest or abdominal wall consistent with routine and traditional clinical practice. The electrical wave activity information recorded by the satellite device 508 may be transmitted to the mother device 506 for further signal processing and wave interference analysis. The mother device 506 is also capable of transmitting electrical wave information to a nearby or remote external base station 600, as illustrated in FIG. 6, with even greater computing power for higher level wave interference analysis. QRS wave data collected by the satellite device 508, as well as a diversity of electrical signals recorded by the mother device 506, may be used to dynamically optimize electrical wave propagation paths for improvement in cardiac structure and function or other related purposes. This same electrical wave activity is dual-purposed for cardiac muscle conduction substrate characterization and quantification, stimulation site selection and wave propagation path assignments, and clinical event prediction modeling, as described herein and in co-pending U.S. application Ser. No. 14/131,868, entitled System and Method for Automated Adjustment of Cardiac Resynchronization Therapy Control Parameters, which is incorporated herein by reference in its entirety.

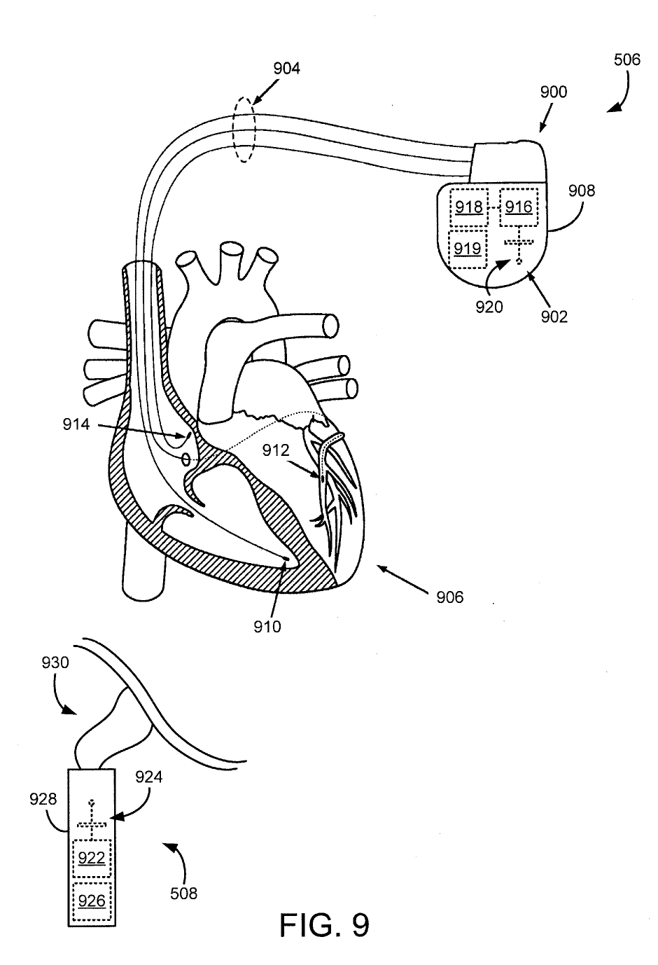

This platform, therefore, integrates near- and far-field human cardiac electrical wave recordings with computational firmware and device-device networking. The mother device 506 may have electrical stimulation and sensing capabilities. For example, referring to FIG. 9, the mother device 506 may include a CIED 900 formed of a pulse generator 902 and intracardiac lead system 904. Portions of the intracardiac lead system 904 may be inserted into the patient's heart 906 by way of the vessels of the upper venous system, such as the superior vena cava; or other methods of access to the heart. The intracardiac lead system 904 includes one or more electrodes configured to produce an electrogram (“EGM”) signal representing cardiac electrical activity sensed at the location of the electrode, between spatially separated electrodes, or between various combinations of electrodes and a housing 908 of the pulse generator 902, or to deliver pacing electrical pulses to the location of the electrode. Optionally, the intracardiac lead system 904 may include one or more electrodes configured to sense physiological parameters, such as cardiac chamber pressure, motion, contractility, vibration or temperature.

The lead system 904 may include one or more intracardiac electrodes 910-914 positioned in, on, or about one or more heart chambers for sensing electrical signals from the patient's heart 906 and delivering pacing pulses to the heart 906. The intracardiac electrodes 910-914, such as those illustrated in FIG. 9, may be used to sense electrical activity in or pace one or more chambers of the heart, including the left ventricle, the right ventricle, the left atrium, and the right atrium. The lead system 904 may include one or more defibrillation electrodes for delivering cardioversion/defibrillation electrical shocks to the heart.

The pulse generator 902 includes circuitry for detecting cardiac arrhythmias and controlling pacing or defibrillation therapy in the form of electrical stimulation pulses or shocks delivered to the heart 906 through the lead system 904. The housing 908 of the pulse generator 902 may also serve as a sensing electrode for recording far-field EGMs in combination with various selectable intracardiac electrodes 910-914. The housing 908 may also enclose a controller 916 formed of a microprocessor in electrical communication with a memory 918 for program and data storage, and a power source 919. Other controller designs will be readily appreciated by those skilled in the art. Also, a mother wireless communications module 920 may be included to facilitate communications with the satellite device 508, which may include its own controller 922 and satellite wireless communications module 924, and communication with external systems, such as the above-described base station 406, 426, 600. Thus, the satellite device 508 may include the controller 922 and satellite wireless communications module 924 and also a power source 926 surrounded by a housing 928 and having electrical leads 930 extending therefrom that are capable of recording body surface electrical wave activity.

The pulse generator 902, acting as the controller, is configured to operate the CIED 900 in a number of programmed modes, including the above-described functionality of the mother device 506. As described above, communications circuitry is also provided for facilitating communication between the controller and an external communication device, such as, for example, a portable or bed-side communication station, patient-carried/worn communication station, or external programmer.

The controller controls the overall operation of the CIED 900 in accordance with programmed instructions stored in memory. More specifically, the sensing circuitry of the CIED 900 generates multiple atrial, ventricular, and far-field EGM signals (which indicate the time course and amplitude of cardiac depolarization that occurs during either an intrinsic or paced beat), alone and in various combinations, from the voltages sensed by the electrodes of a particular channel. The controller interprets the EGM signals sensed from the intracardiac electrodes 910-914, and far-field electrodes formed with the housing 908 of the pulse generator 902, and controls the delivery of pacing electrical pulses in accordance with a programmed pacing mode.

Thus, the above-described CIED 900 forms part of or acts as the mother device 506 to form, with the satellite device 508, a body worn application system for human-computer interaction satisfies the requirement for a workable wireless solution for body networking of cardiac electrical activity. The near-field short range wireless network uses low-power integrated circuits and physiologic sensors to achieve a wireless sensor network. The implanted sensors collect various physiological signals which can be used for therapy titration and clinical event prediction. The physically separate devices 506, 508 can automatically and quickly associate, or “pair” electronically. The mother device 506 may be the lone inquiring (“paging”) device that actively sends inquiry requests. The remote satellite device 508, when enabled, may be configured to operate as a discoverable (“page scanning”) device that can only listen for inquires and send responses. The satellite device 508 and the mother device 506 may have protection features to guarantee exclusively monogamous pairing for therapy delivery, similar to a “Piconet” topology. In this manner, the mother device 506 serves as the “master” and the satellite device 508 functions as the “slave”. The mother device 506 is capable of communicating at longer ranges, such as a bedside monitor and transmitter, thereby connecting the local area body network to the internet for higher level analytics and various reporting applications. The satellite device 508 has similar capabilities for reporting purposes. Unlike other Piconet arrangements, the connection between master and slave may be configured to never be broken unintentionally because the RF link is always maintained since the two devices reside in physical proximity and are never out of range of one another. Thus, body bus pairing connections are maintained until they are deliberately broken by the mother device 506 at the conclusion of a duty cycle, or by external command.

Thus, systems and methods are provided for autonomous, implanted subcutaneous or, submuscular, electrode pair that includes an electrical wave sensor device, or “satellite”, having a conductive jacket, and possessing one or more configurable electrodes capable of recording body surface electrical wave activity, an energy source, electrical waveform processing circuitry, a wireless transmitter, and the capability of wireless pairing with a second physically remote subcutaneous device, and an external base station. The system also includes a second “mother” device having similar capabilities, and also possessing the capability of multipoint cardiac electrical stimulation, a microcontroller capable of interfacing with the first device, a microcontroller also capable of supporting mathematical algorithms for analysis and correlation of wave interference patterns utilizing body surface and intra-cardiac electrical activity, and possessing an energy source. Furthermore, the physically-separate subcutaneous devices are capable of multidirectional RF communication, where RF communication occurs directly between the topologically distributed separate implanted devices through the physical medium of the human body, thereby creating, a local area network within the human body serving as the system bus that transfers data between components of the system.

The present invention has been described in terms of one or more preferred embodiments, and it should be appreciated that many equivalents, alternatives, variations, and modifications, aside from those expressly stated, are possible and within the scope of the invention.

1.-17. (canceled)

18. A guidance method for cardiac resynchronization therapy of a subject's heart in communication with a cardiac implantable electrical device (CIED), the method comprising:
a) acquiring signals representing cardiac electrical activity in the subject's heart using electrodes in electrical communication with an external workstation;
b) identifying a baseline waveform from the acquired cardiac electrical activity signals;
c) acquiring signals representing a QRS fusion wave using the electrodes after CIED stimulation with a first set of wave control parameters;
d) comparing the QRS fusion wave to the baseline waveform;
e) determining a fusion response phenotype based on the comparison in step d);
f) estimating odds of reverse remodeling when delivering cardiac resynchronization therapy using the first set of wave control parameters based on characteristics of the baseline waveform and the QRS fusion wave;
g) repeating steps c) through f) using a second set of wave control parameters; and
h) generating a wave interference library associating each of the first set of wave control parameters and the second set of wave control parameters with their respective QRS fusion waves and estimated odds of reverse remodeling.

19. The guidance method of claim 18, wherein each of the first set of parameters and the second set of parameters include left ventricular electrode site, right ventricular electrode site, and electrical conduction pathway.

20. The guidance method of claim 18, wherein each of the first set of parameters and the second set of parameters include at least one of atrioventricular timing interval, interventricular timing interval, left ventricular electrode site, and electrical conduction pathway.

21. The guidance method of claim 18, wherein the electrodes are body-surface ECG electrodes.

22. The guidance method of claim 18, further comprising identifying an optimal set of wave control parameters from the wave interference library based on the estimated odds of reverse remodeling.

23. The guidance method of claim 18, wherein the fusion response phenotype includes one of an oblique fusion responder phenotype, a cancellation fusion responder phenotype, and a summation fusion non-responder phenotype.

24. The guidance method of claim 18, wherein step f) includes estimating the odds of reverse remodeling based on the fusion response phenotype, a difference in duration between the QRS fusion wave and the baseline waveform, and left ventricle scar in the subject's heart.

25. A guidance method for setting cardiac resynchronization therapy parameters of a cardiac implantable electrical device (CIED) in communication with a subject's heart, the method comprising:
for each of a plurality of stimulation sites,
a) receiving signals indicative of cardiac electrical activity in the heart in response to stimulation at the respective stimulation site with one set of a plurality of sets of wave control parameters,
b) determining a QRS fusion wave contour from the signals, the QRS fusion wave contour being a composition of a wavefront propagated from the left ventricle and an opposing wavefront propagated from the right ventricle,
c) storing the QRS fusion wave contour in relation to the respective stimulation site and the respective set of wave control parameters in a wave interference library, and
d) repeating steps a) through c) using a second set of the plurality of sets of wave control parameters;
selecting a QRS fusion wave contour from the wave interference library indicating the most opposition between the wavefront propagated from the left ventricle and the opposing wavefront propagated from the right ventricle as measured by one of morphology, amplitude, and time duration; and
setting cardiac resynchronization therapy parameters of the CIED using the respective set of wave control parameters and the respective stimulation site associated with the selected QRS fusion wave contour.

26. The guidance method of claim 25, further comprising:
storing the selected QRS fusion wave contour as a template;
receiving new signals indicative of cardiac electrical activity in the heart in response to stimulation with the cardiac resynchronization therapy parameters;
determining an incoming QRS fusion wave contour from the new signals; and
comparing the incoming QRS fusion wave contour to the template.

27. The guidance method of claim 26, further comprising updating the cardiac resynchronization therapy parameters based on the comparison.

28. The guidance method of claim 25, wherein step a) includes receiving the signals from a body surface recording source.

29. The guidance method of claim 25, further comprising estimating odds of reverse remodeling based on delivering cardiac resynchronization therapy parameters using the respective set of wave control parameters and the respective stimulation site associated with each QRS fusion wave contour and storing the odds of reverse remodeling for each QRS fusion wave contour in the wave interference library.

30. The guidance method of claim 25, further comprising storing the cardiac resynchronization therapy parameters in memory of the CIED.

31. The guidance method of claim 25, wherein the stimulation site includes a left ventricle stimulation site.

32. The guidance method of claim 31, wherein step a) includes receiving the signals indicative of cardiac electrical activity in the heart in response to stimulation at the respective stimulation site and at a right ventricle stimulation site with the one set of the plurality of sets of wave control parameters.
